(19) United States
(12) Patent Application Publication
(10) Pub. No.: US 2026/0354745 A1
(43) Pub. Date: Nov. 12, 2026

(54) METHOD FOR ESTIMATING SKIN CONDITION, INFORMATION PROCESSING APPARATUS, AND COMPUTER PROGRAM

(71) Applicants: Shiseido Company, Ltd., Chuo-ku, Tokyo (JP); RIKEN, Wako-shi, Saitama (JP)

(72) Inventors: Chieko MIZUMOTO, Tokyo (JP); Shuhei IMAMURA, Tokyo (JP); Tatsumi TSUJI, Tokyo (JP); Yuki TANAKA, Tokyo (JP); Keiko OTAKE, Tokyo (JP); Ryota WAKI, Tokyo (JP); Yusuke MORITO, Tokyo (JP); Kei MIZUNO, Saitama (JP); Kyosuke WATANABE, Saitama (JP); Yasuyoshi WATANABE, Saitama (JP)

(73) Assignees: Shiseido Company, Ltd., Chuo-ku, Tokyo (JP); RIKEN, Wako-shi, Saitama (JP)

(21) Appl. No.: 19/131,642

(22) PCT Filed: Nov. 22, 2023
(86) PCT No.: PCT/JP2023/042023

§ 371 Date: May 21, 2025

(30) Foreign Application Priority Data
Nov. 22, 2022 (JP) 2022-186377
Aug. 10, 2023 (JP) 2023-131043
Sep. 4, 2023 (JP) 2023-143248

(51) Int. Cl. A61B 5/00

(52) U.S. Cl. CPC A61B 5/441; A61B 5/7246; A61B 5/7278

(57) ABSTRACT

A method for estimating a skin condition includes the steps of: acquiring mind-body data relating to the body and/or mind of a subject of skin condition estimation; estimating the subject's skin condition using a skin model that describes a correlation between the mind-body data and the skin condition, the mind-body data including at least one of liver function related variables, muscle-bone related variables, heart rate and blood pressure related variables, blood oxidation related variables, blood count related variables, lipid metabolism related variables, autonomic function related variables, personal attribute related variables, renal function related variables, body composition related variables, glucose tolerance related variables, electrolyte related variables, cognitive function related variables, immunometabolism related variables, and depression-fatigue-drowsiness related variables.

### TECHNICAL FIELD

The present invention relates to a method for estimating skin condition, an information processing apparatus, and a computer program.

### BACKGROUND

Various methods for estimating skin conditions based on information on the human body are known.

For example, the international publication WO2022-030300 discloses a technology for predicting future skin condition based on hormone balance.

For example, JP 2020-014710 A discloses a technology for estimating skin condition (at least one of pores, wrinkles, pigmentation, and skin color) using as indices of muscle mass (muscle weight per body weight, or muscle volume per volume, trunk (chest and abdomen), lower body, or the combined total of the trunk and the lower body).

### SUMMARY OF INVENTION

### Technical Problem

There are many different factors that interact with each other and affect the skin condition.

However, since the hormone balance in international publication WO2022-030300 and the muscle mass in Japanese Patent Laid-Open No. JP2020-014710 are only some of the indices of the human body, estimations based on hormone balance or muscle mass are not necessarily appropriate.

An object of the present invention is to more appropriately estimate the skin condition and factors that define the skin condition.

### Solution to Problem

One aspect of the present invention is a method for estimating a skin condition, the method comprising the steps of:
acquiring mind-body data relating to the body and/or mind of a subject of skin condition estimation; and
estimating the subject's skin condition using a skin model that describes a correlation between the mind-body data and the skin condition, the mind-body data including at least one of liver function related variables, muscle-bone related variables, heart rate and blood pressure related variables, blood oxidation related variables, blood count related variables, lipid metabolism related variables, autonomic function related variables, personal attribute related variables, renal function related variables, body composition related variables, glucose tolerance related variables, electrolyte related variables, cognitive function related variables, immunometabolism related variables, and depression-fatigue-drowsiness related variables.

### BRIEF DESCRIPTION OF DRAWINGS

FIG. 1 is a block diagram showing a configuration of an information processing system according to the present embodiment.

FIG. 2 is a functional block diagram of the information processing system of FIG. 1.

FIG. 3 is an explanatory diagram of an overview of the present embodiment.

FIG. 4 is a diagram showing the data structure of a subject database according to the present embodiment.

FIG. 5 is a diagram showing a data structure of an estimation log database according to the present embodiment.

FIG. 6 is a schematic diagram showing a configuration of a skin model according to the present embodiment.

FIG. 7 is a sequence diagram of information processing according to the present embodiment.

FIG. 8 is a diagram showing examples of screens displayed in the information processing of FIG. 7.

FIG. 9 is a diagram illustrating an overview of the first modification.

FIG. 10 is a sequence diagram of information processing according to the first modification.

FIG. 11 is a diagram illustrating an overview of the second modification.

FIG. 12 is a sequence diagram of information processing according to the second modification.

FIG. 13 is a diagram illustrating an overview of the third modification.

FIG. 14 is a schematic diagram showing a configuration of the first skin model according to the third modification.

FIG. 15 is a schematic diagram showing a configuration of the second skin model according to the third modification.

FIG. 16 is a sequence diagram of information processing in the third modification.

FIG. 17 is diagram showing examples of screens displayed in the information processing of FIG. 16.

FIG. 18 is a diagram illustrating an overview of the fourth modification.

FIG. 19 is a sequence diagram of information processing in the fourth modification.

FIG. 20 is a diagram illustrating an overview of the fifth modification.

FIG. 21 is a sequence diagram of information processing in the fifth modification.

### DESCRIPTION OF EMBODIMENTS

Hereinafter, the present embodiment is described in detail based on the drawings.

Note that, in the drawings for describing the present embodiments, the same components are denoted by the same reference sign in principle, and the repetitive description thereof is omitted.

### (1) Configuration of Information Processing System

The configuration of information processing system will be described.

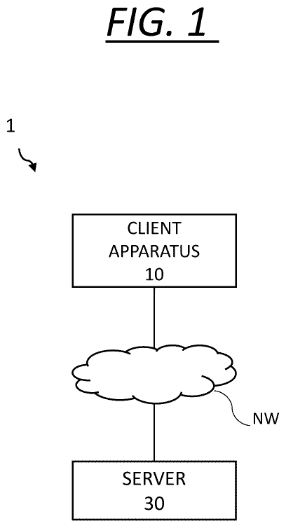

FIG. 1 is a block diagram showing the configuration of the information processing system according to the present embodiment.

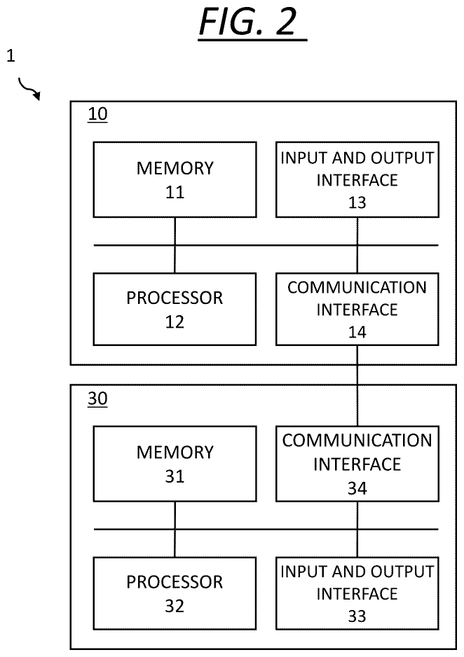

FIG. 2 is a functional block diagram of the information processing system of FIG. 1.

As shown in FIG. 1, the information processing system 1 includes a client apparatus 10 and a server 30.

The client apparatus 10 and the server 30 are connected via a network (for example, the Internet or an intranet) NW.

The client apparatus 10 is a computer (an example of an “information processing apparatus”) that transmits a request to the server 30.

The client apparatus 10 is, for example, a smart phone, a tablet terminal, or a personal computer.

The server 30 is a computer (an example of an “information processing apparatus”) that provides the client apparatus 10 with a response in response to the request sent from the client apparatus 10.

The server 30 is, for example, a web server.

### (1-1) Configuration of Client Apparatus

A configuration of the client apparatus 10 will be described.

As shown in FIG. 2, the client apparatus 10 includes a memory 11, a processor 12, an input and output interface 13, and a communication interface 14.

The memory 11 is configured to store programs and data.

The memory 11 is, for example, a combination of a ROM (read only memory), a RAM (random access memory), and a storage (for example, a flash memory or a hard disk).

Programs include, for example, the following programs:
OS (Operating System) program; and
Programs of applications that execute information processing (for example, web browsers).

The data includes, for example, the following data:
Databases referenced in information processing; and
Data acquired by executing information processing (that is, the result of executing information processing).

The processor 12 is configured to implement the functions of the client apparatus 10 by activating programs stored in the memory 11.

The processor 12 is, for example, a CPU (Central Processing Unit), an ASIC (Application Specific Integrated Circuit), an FPGA (Field Programmable Gate Array), or a combination thereof.

The input and output interface 13 is configured to acquire a user's instruction from an input device connected to the client apparatus 10 and output information to an output device connected to the client apparatus 10.

The input device is, for example, a keyboard, a pointing device, a touch panel, or a combination thereof.

The output device is, for example, a display.

The communication interface 14 is configured to control communications between the client apparatus 10 and the server 30.

### (1-2) Configuration of Server

A configuration of the server 30 will be described.

As shown in FIG. 2, the server 30 includes a memory 31, a processor 32, an input and output interface 33, and a communication interface.

The memory 31 is configured to store a program and data.

The memory 31 is, for example, a combination of ROM, RAM, and storage (for example, flash memory or hard disk).

Programs include, for example, the following programs:
OS program; and
Programs of applications that execute information processing.

The data includes, for example, the following data:
Databases referenced in information processing; and
Result of executing information processing.

The processor 32 is configured to implement the functions of the server 30 by activating programs stored in the memory 31.

Processor 32 is, for example, a CPU, ASIC, FPGA, or a combination thereof.

The input and output interface 33 is configured to acquire user's instruction from an input device connected to the server 30 and to output information to output devices connected to the server 30.

The input device is, for example, a keyboard, a pointing device, a touch panel, or a combination thereof.

The output device is, for example, a display.

The communication interface 34 is configured to control communications between the server 30 and the client apparatus 10.

### (2) Overview of Embodiment

An overview of the present embodiment will be described.

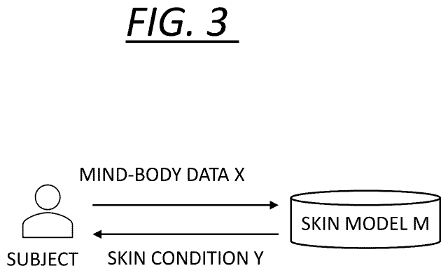

FIG. 3 is a diagram illustrating an overview of the present embodiment.

As shown in FIG. 3, in the skin model M of the present embodiment, the correlation between the mind-body data X and the skin condition Y is described.

When the subject's mind-body data X is provided to a skin model M, a skin condition Y corresponding to the mind-body data X is estimated.

The estimated skin condition Y is presented to the subject.

### (3) Database

The data structure of the database in the present embodiment will be described.

### (3-1) Subject Database

The data structure of the subject database of the present embodiment will be described.

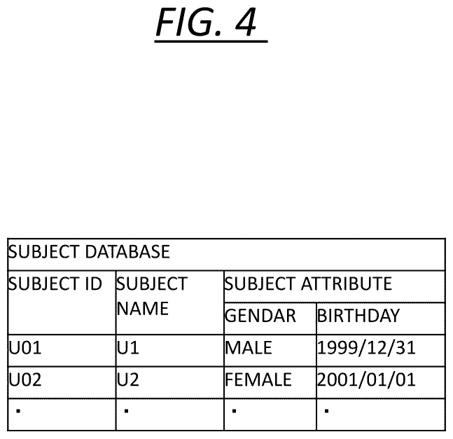

FIG. 4 is a diagram showing the data structure of the subject database of the present embodiment.

As shown in FIG. 4, subject information is stored in the subject database.

The subject information is information on the subject.

The subject database includes a “subject ID” field, and a “subject name” field, and a “subject attribute” field.

Each field is associated with another.

The “Subject ID” field stores subject identification information.

The subject identification information is information that identifies the subject.

The “subject name” field stores subject name information.

The subject name information is information on the subject's name.

The “subject attribute” field stores subject attribute information.

The subject attribute information is information on the subject's attributes (an example of “mind-body data”).

The “subject attribute” field includes a “gender” field and a “birthday” field.

The “gender” field stores gender information.

The gender information is information on the gender of the subject.

The “birthday” field stores birthday information.

The birthday information is information on the subject's birthday.

Based on a combination of the birthday information and the date on which the information processing is executed, the age of the subject on the date on which the information processing is performed can be specified.

### (3-2) Estimation Log Database

The data structure of the estimation log database of the present embodiment will be described.

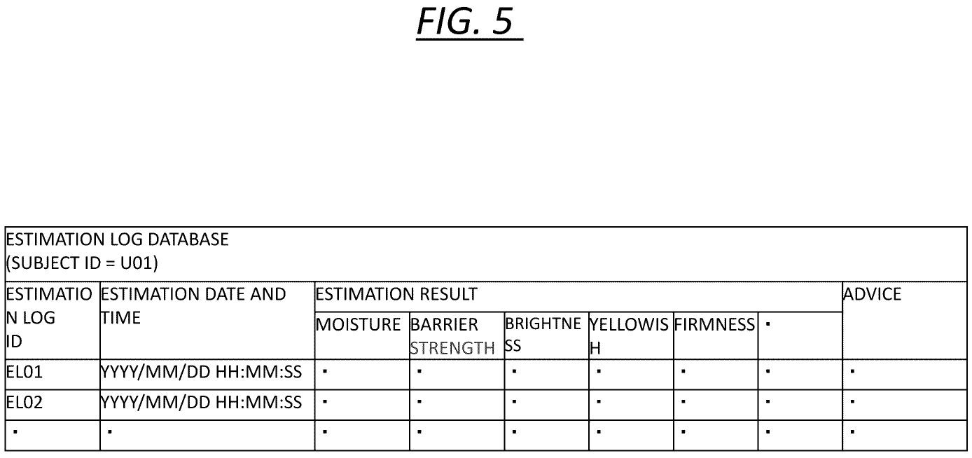

FIG. 5 is a diagram showing the data structure of the estimation log database of the present embodiment.

As shown in FIG. 5, the estimation log database stores estimation log information.

The estimation log information is information on the history of estimation results of the subject's skin condition.

The estimation log database includes an “estimation log ID” field, and a “estimation date and time” field, an “estimation result” field, and an “advice” field.

Each field is associated with another.

The inferred log database is associated with subject identification information.

The “estimation log ID” field stores estimation log identification information.

The estimation log identification information is information for identifying the history of the estimation result.

The “estimation date and time” field stores estimation date and time information.

The estimation date and time information is information on the date and time when the estimation has been performed.

The “estimation result” field stores estimation result information.

The estimation result information is information on the estimation result of the skin condition.

The “estimation result” field includes multiple skin index fields.

The skin index field includes, for example:
“Moisture” field: The estimation result of the skin moisture content is stored;
“Barrier strength” field: The estimation result of skin's barrier strength is stored;
“Brightness” field: The estimation result of skin brightness is stored;
“Yellowness” field: The estimation result of yellowness of the skin is stored;
“Firmness” field: The estimation result of skin firmness is stored.
“Melanin” field (not shown): The estimation result of skin melanin is stored;
“Hemoglobin” field (not shown): The estimation result of skin hemoglobin is stored;
“Vividness” field (not shown): The estimated skin vividness is stored;
“Redness” field (not shown): The estimation result of skin redness is stored;
“Texture” field (not shown): The estimation result of skin texture is stored;
“Wrinkles” field (not shown): The estimation result of skin wrinkles is stored; and
“Skin spots” field (not shown): The estimation result of skin pigmentations is stored.

The “advice” field stores advice information.

The advice information is information on advice that is provided to the subject and is determined according to the estimation result of the skin condition.

### (4) Skin Model

The skin model of the present embodiment will be described.

### (4-1) Architecture of the Skin Model

The architecture of the skin model will now be described.

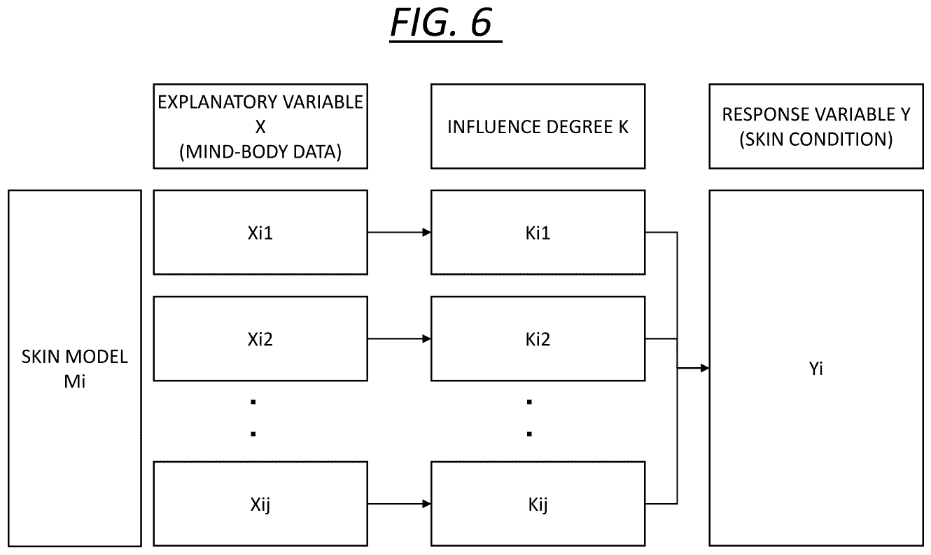

FIG. 6 is a schematic diagram showing the architecture of a skin model according to the present embodiment.

As shown in FIG. 6, a skin model Mi is generated for each index of a skin condition (hereinafter referred to as a “skin index”).

The skin model Mi is stored in the memory 31.

The skin indices are classified into the following skin index groups, for example:
Skin texture index group (hereinafter referred to as “texture index group”);
Skin structure index group (hereinafter referred to as “structure index group”); and
Skin color index group (hereinafter referred to as “color index group”).

The texture index group includes, for example, the following indexes:
Index related to skin moisture (hereinafter referred to as “moisture related index”); and
Index related to skin surface morphology (hereinafter referred to as “surface morphology related index”).

The moisture related index includes, for example, the following indices:
Skin moisture: and
Skin barrier strength.

The surface morphology related index includes, for example, skin texture.

The structural index group includes, for example, the following indices:
Index related to skin elasticity (hereinafter referred to as “elasticity related index”); and
Index related to skin wrinkles (hereinafter referred to as “wrinkle related indexes”).

The elasticity related index includes, for example, skin firmness.

The wrinkle related index includes, for example, skin wrinkles.

The color index group includes, for example, the following indices:
Index related to skin color (hereinafter referred to as “skin color related index”); and
Index related to spots (hereinafter referred to as “spot related index”).

The skin color related index includes, for example, the following indices:
Melanin in the skin;
Hemoglobin in the skin;
Skin brightness;
Skin vividness;
Skin redness; and
Skin yellowness.

The skin spots related indices include, for example, skin pigmentations.

As shown in FIG. 6, an explanatory variable Xij, an influence degree Kij, and a target variable Yi are defined for the skin model Mi.

The argument i (i is a natural number) is an identifier of the skin index.

The argument j (j is a natural number) is an identifier of a variable related to the mind-body data.

The explanatory variables Xij are variables related to the mind-body data.

The influence degree Kij is a parameter of a variable related to the mind-body data.

The influence degree Kij indicates the influence degree of the mind-body data on the skin condition.

The objective variable Yi is the skin condition.

The skin condition is expressed in at least one of the following ways, for example:
Value;
Level; and
Classification.

### (4-2) Mind-Body Data

The mind-body data of the present embodiment will now be described.

Mind-body data is data relating to the human body and/or mind.

Variables relating to mind-body data are classified into one of the following related variables.

The related variables are a set of variables related to the mind-body data.
Liver function related variables;
Muscle-bone related variables;
Heart rate and blood pressure related variables;
Blood oxidation related variables;
Blood count related variables;
Lipid metabolism related variables;
Autonomic function related variables;
Personal attribute related variables;
Renal function related variables;
Body composition related variables;
Glucose tolerance related variables;
Electrolyte related variables;
Cognitive function related variables;
Immunometabolism related variables; and
Depression-fatigue-drowsiness related variables.

The liver function related variables are variables related to liver function.

The liver function related variables preferably include:
ALT (GPT) in blood;
AST (GOT) in blood;
ALP in blood;
γ-GTP in blood;
Indirect bilirubin in blood;
Total bilirubin in blood; and
Direct bilirubin in blood, or the like.

The muscle-bone related variables are variables related to muscles and bones.

The muscle-bone related variables preferably include:
Grip strength; and
Bone density, or the like.

The heart rate and blood pressure related variables are variables related to heart rate and blood pressure.

The heart rate and blood pressure related variables preferably include:
Diastolic blood pressure;
Systolic blood pressure; and
Heart rate (HR), or the like.

The blood oxidation related variables are variables related to blood oxidation.

The blood oxidation related variable preferably includes:
Biological Antioxidant Potential (BAP) in blood;
Reactive oxygen metabolites (d-ROMs) in blood; and
Oxidation Stress Index (OSI), or the like.

The blood count related variables are variables related to blood count.

The blood count related variables preferably includes:
Hematocrit in blood;
Hemoglobin in blood;
Platelets in blood;
Red blood cell in blood;
White blood cell in blood;
Mean corpuscular hemoglobin concentration in blood;
Mean corpuscular hemoglobin in blood; and
Mean corpuscular volume in blood, or the like.

The lipid metabolism related variables are variables related to lipid metabolism.

The lipid metabolism related variables preferably include:
HDL cholesterol in blood;
LDL cholesterol in blood;
Ratio of LDL cholesterol in blood to HDL cholesterol in blood;
Total cholesterol in blood; and
Neutral fat in blood, or the like.

The autonomic function related variables are variables related to the autonomic nervous function.

The autonomic function related variables preferably include:
Autonomic indices HF (high-frequency component of the power spectrum in the time series variation of heartbeat intervals);
Autonomic indices LF (low-frequency component of the power spectrum in the time series variation of heartbeat intervals); and
Ratio of autonomic indices HF to autonomic indices LF, or the like.

The personal attribute related variables are variables related to personal attributes.

The personal attribute related variables preferably include:
Exercise habits (for example, average daily steps);
Sleep time value;
Duration of fatigue;
Average working time;
Medical history;
Smoking history;
Regular medication; and
Marital history, or the like.

The renal function related variables are variables related to renal function.

The renal function related variables preferably include:
Estimated glomerular filtration rate (eGFR); and
Urea nitrogen in blood, or the like.

The body composition related variables are variables related to body composition.

The body composition related variables preferably include:
BMI (Body Mass Index);
Muscle mass;
Height;
Body fat percentage;
Body weight; and
Skeletal muscle mass, or the like.

The glucose tolerance related variables are variables related to glucose tolerance.

The glucose tolerance related variables preferably include:
Hemoglobin A1c (HbA1c) in blood; and
Glucose in blood, or the like.

The electrolyte related variables are variables related to electrolytes.

The electrolyte related variable preferably includes:
Kalium in blood;
Chloride in blood;
Natrium in blood; and
Ratio of natrium in blood to kalium in blood, or the like.

The cognitive function related variables are variables related to cognitive function and are acquired from the performance on tasks in a cognitive function test.

The cognitive function related variables preferably include:
Reaction time for cognitive tasks;
Correct answer rate of cognitive tasks;
Incorrect answer rate of cognitive tasks; and
Number of completed cognitive tasks, or the like.

The immunometabolism related variables are variables related to immunometabolism.

The immunometabolism related variables preferably include:
High-sensitivity CRP (C-reactive protein) in blood;
Amylase in blood;
Albumin in blood;
Creatine kinase in blood;
Cortisol in blood;
Total protein in blood;
Lactate dehydrogenase in blood:
Uric acid in blood;
IgE in blood;
IgG in blood; and
IgA in blood, or the like.

The depression-fatigue-drowsiness related variables are variables related to depression, fatigue, and drowsiness, and are variables acquired by questionnaire survey.

The depression-fatigue-drowsiness related variables preferably include:
Fatigue assessment scale;
Depression-anxiety assessment scale;
Motivation assessment scale;
Drowsiness assessment scale; and
Life performance status assessment scale, or the like.

### (4-3) Method for Generating Skin Model

A method for generating a skin model according to the present embodiment will now be described.

In the present embodiment, a skin model is generated using a combination of mind-body data and skin conditions of a plurality of subjects as training data, for example, by using any one of the following methods.

Examples include simple regression analysis, multiple regression analysis, Elastic NET, PLS (Partial Least Squares), neural networks, and DNN (Deep Neural Network).

### (4-4) Examples of Skin Models

A specific example of the skin model of the present embodiment will be described.

### (4-4-1) Specific Examples of Skin Models of Texture Index Groups

A specific example of a skin model of the texture index group of the present embodiment will be described.

As an example of a skin model for a moisture related index in the texture index group, a skin model M1 for estimating a skin index “skin moisture” Y1 includes, for example, at least one of the following variables:
Fatigue assessment scale;
Drowsiness assessment scale;
Depression-anxiety assessment scale;
Body weight;
BMI: Body Mass Index;
Body fat percentage;
Exercise habits;
Duration of fatigue;
Uric acid in blood;
Cortisol in blood;
Reaction time for cognitive tasks;
Kalium in blood;
Natrium in blood;
Heart rate (HR);
LDL cholesterol in blood;
Neutral fat in blood;
Red blood cell in blood;
White blood cell in blood;
Platelets in blood;
Reactive oxygen metabolites (d-ROMs) in blood;
Biological antioxidant potential (BAP) in blood;
Oxidation Stress Index (OSI);
Total bilirubin in blood;
Indirect bilirubin in blood;
Hematocrit in blood;
Grip strength;
IgA in blood; and
Hemoglobin A1C (HbA1c) in blood.

As an example, the skin index “skin moisture” Y1 is affected by each variable as follows:
Hematocrit in blood shows a positive correlation with the skin index “skin moisture” Y1 (that is, the higher the hematocrit in blood, the better the skin index “skin moisture” Y1).

The fatigue assessment scale shows a negative correlation with the skin index “skin moisture” Y1 (that is, the higher the fatigue, the worse the skin index “skin moisture” Y1).

The depression-anxiety assessment scale shows a negative correlation with the skin index “skin moisture” Y1 (that is, the stronger the depression and anxiety, the worse the skin index “skin moisture” Y1).

The skin model M2 for estimating the skin index “skin barrier strength” Y2, which is a moisture related index in the texture index group, includes, for example, at least one of the following variables.

Here, barrier strength refers to an index that represents the strength of the skin's moisturizing function, such as the inverse of transepidermal water loss:
Fatigue assessment scale;
Drowsiness assessment scale;
Depression-anxiety assessment scale;
Muscle mass;
Exercise habits;
Duration of fatigue;
Uric acid in blood;
Reaction time for cognitive tasks;
Kalium in blood;
Hemoglobin A1C (HbA1c) in blood;
Glucose in blood;
Systolic blood pressure;
White blood cell in blood;
Platelets in blood;
Reactive oxygen metabolites (d-ROMs) in blood;
Biological antioxidant potential (BAP) in blood;
Oxidation Stress Index (OSI);
γ-GTP in blood;
Direct bilirubin in blood;
Chloride in blood; and
ALT (GPT) in blood.

As an example, the skin index “skin barrier strength” Y2 is influenced by each variable as follows.

The fatigue assessment scale shows a negative correlation with the skin index “skin barrier strength” Y2 (that is, the stronger the fatigue, the weaker the “skin barrier strength” Y2):
Depression-anxiety assessment scale shows a negative correlation with the skin index “skin barrier strength” Y2 (that is, the stronger the depression and anxiety, the weaker the “skin barrier strength” Y2).

As an example of a skin model of a surface morphology related index of the texture index group, a skin model M3 for estimating a skin index “skin texture” Y3 includes, for example, at least one of the following variables:
Body weight;
Lactate dehydrogenase in blood;
Amylase in blood;
Autonomic indices LF;
Mean corpuscular volume in blood;
Platelets in blood;
ALT (GPT) in blood;
ALP in blood;
Ratio of natrium in blood to kalium in blood;
IgE in blood;
Ratio of LDL cholesterol in blood to HDL cholesterol in blood; and
Skeletal muscle mass.

### (4-4-2) Specific Examples of Skin Models of Structural Index Groups

A specific example of a skin model of the structure index group of the present embodiment will be described.

As an example of a skin model for an elasticity related index of the structure index group, a skin model M4 for estimating a skin index “skin firmness” Y4 includes, for example, at least one of the following variables.
Fatigue assessment scale;
Drowsiness assessment scale;
Depression-anxiety assessment scale;
Body weight;
Muscle mass;
Sleep time value;
Exercise habits;
Lactate dehydrogenase in blood;
Hemoglobin A1C (HbA1c) in blood;
Glucose in blood;
Systolic blood pressure;
Estimated glomerular filtration rate (eGFR);
Autonomic indices HF and autonomic indices LF;
Hemoglobin in blood;
Hematocrit in blood;
White blood cell in blood
Platelets in blood;
Reactive oxygen metabolites (d-ROMs) in blood;
AST (GOT) in blood;
Direct bilirubin in blood;
Grip strength;
ALP in blood;
Reactive oxygen metabolites (d-ROMs) in blood; and
Body fat percentage.

As an example, the skin index “skin firmness” Y4 is influenced by each variable as follows:
AST in blood shows a positive correlation with the skin index “skin firmness” Y4 (that is, the higher the AST in blood, the better the skin index “skin firmness” Y4);
The fatigue assessment scale shows a positive correlation with the skin index “skin firmness” Y4 (that is, the higher the fatigue, the more the skin index “skin firmness” Y4 improves); and
It shows a positive correlation with the depression-anxiety assessment scale skin index “skin firmness” Y4 (that is, the higher the depression and anxiety, the more the skin index “skin firmness” Y4 improves).

As an example of a skin model of a structure related index of the structure index group, a skin model M5 for estimating a skin index “skin wrinkles” Y5 includes, for example, at least one of the following variables.
Body weight;
Systolic blood pressure; and
Average working time.

### (4-4-3) Specific Examples of Skin Models of Color Index Groups

A specific example of the skin model of the color index group of the present embodiment will be described.

As an example of a skin model for a skin color related index in the color index group, a skin model M6 for estimating a skin index “skin melanin” Y6 includes, for example, at least one of the following variables:
Fatigue assessment scale;
Drowsiness assessment scale;
Depression-anxiety assessment scale;
Body weight;
Exercise habits;
Uric acid in blood;
Reaction time for cognitive tasks;
Kalium in blood;
Hemoglobin A1C (HbA1c) in blood;
Glucose in blood;
Autonomic indices HF;
Hemoglobin in blood;
Hematocrit in blood;
Mean corpuscular hemoglobin in blood;
Mean corpuscular hemoglobin concentration in blood;
White blood cell in blood;
Platelets in blood;
Reactive oxygen metabolites (d-ROMs) in blood;
AST (GOT) in blood;
γ-GTP in blood;
ALT (GPT) in blood;
Total bilirubin in blood;
Direct bilirubin in blood;
Ratio of autonomic indices HF and autonomic indices LF; and
Red blood cell in blood.

As an example of a skin model for a skin color related index in the color index group, a skin model M7 for estimating a skin index “skin hemoglobin” Y7 includes, for example, at least one of the following variables:
Fatigue assessment scale;
Drowsiness assessment scale;
Depression-anxiety assessment scale;
BMI: Body Mass Index;
Body fat percentage;
Exercise habits;
Duration of fatigue;
Reaction time for cognitive tasks;
Kalium in blood;
Glucose in blood;
Systolic blood pressure;
Total cholesterol in blood;
Neutral fat in blood;
Hematocrit in blood;
Mean corpuscular volume in blood;
White blood cell in blood;
Platelets in blood;
Reactive oxygen metabolites (d-ROMs) in blood;
Biological antioxidant potential (BAP) in blood;
Oxidation Stress Index (OSI);
γ-GTP in blood; and
Direct bilirubin in blood.

As an example of a skin model of a skin color related index in the color index group, a skin model M8 for estimating a skin index “skin brightness” Y8 includes, for example, at least one of the following variables:
Motivation assessment scale;
Body weight;
Muscle mass;
BMI: Body Mass Index;
Body fat percentage;
Sleep time value;
Average working time;
Exercise habits;
Lactate dehydrogenase in blood;
Uric acid in blood;
High-sensitivity CRP (C-reactive protein) in blood;
Reaction time for cognitive tasks;
Kalium in blood;
Systolic blood pressure;
Diastolic blood pressure;
Estimated glomerular filtration rate (eGFR);
Urea nitrogen in blood;
Ratio of autonomic indices HF and autonomic indices LF;
HDL cholesterol in blood;
LDL cholesterol in blood;
Total cholesterol in blood;
Neutral fat in blood;
Mean corpuscular hemoglobin concentration in blood;
White blood cell in blood;
Platelets in blood;
Reactive oxygen metabolites (d-ROMs) in blood;
Biological antioxidant potential (BAP) in blood;
Oxidation Stress Index (OSI); and
Grip strength.

As an example, the skin index “skin brightness” Y8 is influenced by each variable as follows:
Hematocrit in blood shows a negative correlation with the skin index “skin brightness” Y8 (that is, the higher the hematocrit in blood, the worse the skin index “skin brightness” Y8).

As an example of a skin model for a skin color related index in the color index group, a skin model M9 for estimating a skin index “skin vividness” Y9 includes, for example, at least one of the following variables:
Fatigue assessment scale;
Drowsiness assessment scale;
Depression-anxiety assessment scale;
Exercise habits;
Duration of fatigue;
Uric acid in blood;
Reaction time for cognitive tasks;
Kalium in blood
Hemoglobin A1c (HbA1c) in blood;
Glucose in blood;
Hemoglobin in blood;
Hematocrit in blood;
White blood cell in blood;
Platelets in blood;
Reactive oxygen metabolites (d-ROMs) in blood;
AST (GOT) in blood;
γ-GTP in blood;
ALT (GPT) in blood; and
Direct bilirubin in blood.

As an example of a skin model for a skin color related index in the color index group, a skin model M10 for estimating a skin index “skin redness” Y10 includes, for example, at least one of the following variables:
Fatigue assessment scale;
Drowsiness assessment scale;
Depression-anxiety assessment scale;
Body fat percentage;
Exercise habits;
Duration of fatigue;
Uric acid in blood;
Reaction time for cognitive tasks;
Kalium in blood;
Hemoglobin A1C (HbA1c) in blood;
Glucose in blood;
Systolic blood pressure;
Hematocrit in blood;
White blood cell in blood;
Platelets in blood;
Reactive oxygen metabolites (d-ROMs) in blood;
Biological antioxidant potential (BAP) in blood;
Oxidation Stress Index (OSI);
γ-GTP in blood;
ALT (GPT) in blood;
Direct bilirubin in blood; and
AST (GOT) in blood.

As an example, the skin index “skin redness” Y10 is influenced by each variable as follows.
Blood ALT shows a positive correlation with the skin index “skin redness” Y10 (that is, the higher the blood ALT, the better the skin index “skin redness” Y10).

The fatigue assessment scale shows a positive correlation with the skin index “skin redness” Y10 (that is, the stronger the fatigue, the higher the skin index “skin redness” Y10):
It shows a positive correlation with the depression-anxiety assessment scale skin index “skin redness” Y10 (that is, the stronger the depression and anxiety, the more the skin index “skin redness” Y10 improves).

As an example of a skin model of a skin color related index of the color index group, a skin model M11 for estimating a skin index “yellowness of the skin” Y11 includes, for example, at least one of the following variables.
Body weight;
BMI: Body Mass Index;
Body fat percentage;
Exercise habits;
Grip strength;
Total protein in blood;
Uric acid in blood;
High-sensitivity CRP (C-reactive protein) in blood;
Albumin in blood;
Reaction time for cognitive tasks;
Kalium in blood;
Hemoglobin A1C (HbA1c) in blood;
Systolic blood pressure;
Diastolic blood pressure;
Autonomic indices HF;
LDL cholesterol in blood;
Total cholesterol in blood;
Neutral fat in blood;
Hemoglobin in blood;
Mean corpuscular hemoglobin in blood;
Mean corpuscular hemoglobin concentration in blood;
Red blood cell in blood;
White blood cell in blood;
Platelets in blood;
Reactive oxygen metabolites (d-ROMs) in blood;
Oxidation Stress Index (OSI);
AST (GOT) in blood;
γ-GTP in blood;
ALT (GPT) in blood;
Total bilirubin in blood;
Direct bilirubin in blood;
Indirect bilirubin in blood; and
Hematocrit in blood.

As an example, the skin index “yellowness of the skin” Y11 is influenced by each variable as follows:
Blood ALT shows a positive correlation with the skin index “yellowing of the skin” Y11 (that is, the higher the blood ALT, the better the skin index “yellowing of the skin” Y11);
AST in blood shows a positive correlation with the skin index “yellowing of skin” Y11 (that is, the higher the AST in blood, the better the skin index “yellowing of skin” Y11); and
Hematocrit in blood shows a negative correlation with the skin index “skin yellowness” Y11 (that is, the higher the hematocrit in blood, the worse the skin index “skin yellowness” Y11).

As an example of a skin model of a skin color related index of the color index group, a skin model M12 for estimating a skin index “skin spots” Y12 includes, for example, at least one of the following variables:
Drowsiness assessment scale;
BMI: Body Mass Index;
Creatine kinase in blood;
High-sensitivity CRP (C-reactive protein) in blood;
Amylase in blood;
Reaction time for cognitive tasks;
Diastolic blood pressure;
HDL cholesterol in blood;
Red blood cell in blood; and
IgG in blood.

For example, in the case of moisture related index (as an example, the skin index “skin moisture” Y1 and the skin index “skin barrier strength” Y2), the following can be listed as variables with high influence.

In the skin index “skin moisture” Y1, the influence of BMI (Body Mass Index), biological antioxidant potential in blood, body fat percentage, hematocrit in blood, drowsiness assessment scale, blood IgA, kalium in blood, hemoglobin A1c (HbA1c) in blood, fatigue assessment scale, and cortisol in blood is high.

It is preferable that the skin model M1 includes one or more of BMI (Body Mass Index), antioxidant potential value in blood, body fat percentage, hematocrit in blood, drowsiness assessment scale, blood IgA, kalium in blood, hemoglobin A1c (HbA1c) in blood, fatigue assessment scale, and cortisol in blood.

For the skin index “skin barrier strength” Y2, chloride in blood, ALT (GPT) in blood, hemoglobin A1c (HbA1c) in blood, reactive oxygen metabolites (d-ROMs) in blood, γ-GTP in blood, platelets in blood, glucose in blood, and drowsiness assessment scale have a high influence degree.

It is preferable that the skin model M2 includes one or more of chloride in blood, ALT (GPT) in blood, hemoglobin A1c (HbA1c) in blood, reactive oxygen metabolites (d-ROMs) in blood, γ-GTP in blood, platelets in blood, glucose in blood, and drowsiness assessment scale.

For example, in the case of a surface morphology related index (one example is the skin index “skin texture” Y3), the following can be listed as highly influential variables.

In the skin index “skin texture” Y3, the influence of IgE in blood, the ratio of natrium in blood to kalium in blood, autonomic indices LF, and ALP in blood is high.

The skin model M3 preferably contains one or more of IgE in blood, the ratio of natrium in blood to kalium in blood, the autonomic indices LF, and ALP in blood.

For example, in the case of an elasticity related index (as an example, the skin index “skin firmness” Y4), the following can be listed as variables with a high influence degree.

For the skin index “skin firmness” Y4, the influence of reactive oxygen metabolites (d-ROMs) in blood, direct bilirubin in blood, AST (GOT) in blood, ALP in blood, lactate dehydrogenase in blood, oxidative stress level in blood (d-ROMs: reactive oxygen metabolites), body fat percentage, ratio of autonomic indices HF to autonomic indices LF, glucose in blood, white blood cell in blood, and grip strength is high.

It is preferable that the skin model M4 includes one or more of the following: reactive oxygen metabolites (d-ROMs) in blood, direct bilirubin in blood, AST (GOT) in blood, ALP in blood, lactate dehydrogenase in blood, oxidative stress level in blood (d-ROMs: reactive oxygen metabolites), body fat percentage, the ratio of autonomic indices HF to autonomic indices LF, glucose in blood, white blood cell in blood, and grip strength.

For example, in the case of skin color related indices (as an example, the skin index “skin melanin” Y6, the skin index “skin brightness” Y8, and the skin index “skin redness” Y10), the following can be listed as highly influential variables:

In the skin index “skin melanin” Y6, ratio of the autonomic indices HF to the autonomic indices LF, depression-anxiety assessment scale, drowsiness assessment scale, uric acid in blood, glucose in blood, total bilirubin in blood, ALT (GPT) in blood, white blood cell in blood, red blood cell in blood, and reactive oxygen metabolites (d-ROMs) in blood had a high influence.

It is preferable that the skin model M4 includes one or more of the following: the ratio of the autonomic indices HF to the autonomic indices LF, depression-anxiety assessment scale, drowsiness assessment scale, uric acid in blood, glucose in blood, total bilirubin in blood, ALT (GPT) in blood, white blood cell in blood, red blood cell in blood, and reactive oxygen metabolites (d-ROMs) in blood.

In the skin index “skin brightness” Y8, the influence of BMI (Body Mass Index), white blood cell in blood, urea nitrogen in blood, uric acid in blood, body fat percentage, platelets in blood, systolic blood pressure, neutral fat in blood, kalium in blood, and motivation assessment scale is high.

It is preferable that model M8 includes one or more of BMI (Body Mass Index), white blood cell in blood, urea nitrogen in blood, uric acid in blood, body fat percentage, platelets in blood, systolic blood pressure, neutral fat in blood, kalium in blood, and motivation assessment scale.

For the skin index “skin redness” Y10, the influence of body fat percentage, fatigue assessment scale, white blood cell in blood, depression-anxiety assessment scale, AST (GOT) in blood, hematocrit in blood, platelets in blood, reactive oxygen metabolites (d-ROMs) in blood, uric acid in blood, and drowsiness assessment scale is high.

Furthermore, it is preferable that each skin model uses indices that can be acquired non-invasively, or indices that can be acquired through a medical checkup or the like.

Indices that can be acquired non-invasively are not particularly limited as long as they can generally be acquired non-invasively, and examples include BMI (Body Mass Index), sleep assessment scale, fatigue assessment scale, grip strength, body fat percentage, depression-anxiety assessment scale, motivation assessment scale, and systolic blood pressure, or the like.

Indices that can be acquired from health checkups, etc. are not particularly limited as long as they can be acquired from general health checkups, medical examinations, etc., and include blood antioxidant potential (BAP: Biological Antioxidant Potential), hematocrit in blood, reactive oxygen metabolites (d-ROMs) in blood, kalium in blood, hemoglobin in blood, cortisol in blood, IgE in blood, autonomic indices LF, and the ratio of the autonomic indices HF to the autonomic indices LF, or the like.

### (5) Information Processing

The information processing of the present embodiment will be described.

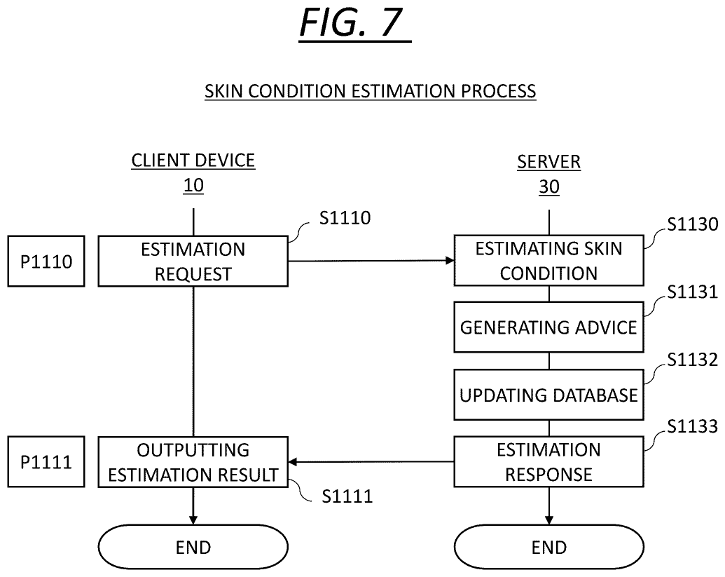

FIG. 7 is a sequence diagram of information processing of the present embodiment.

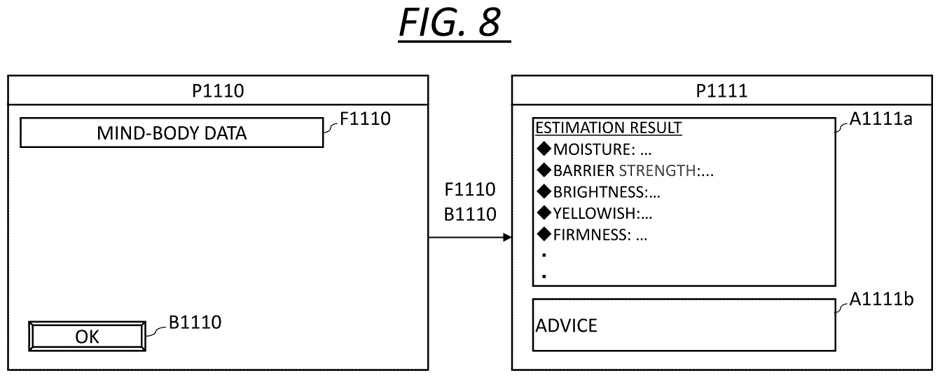

FIG. 8 is a view showing an example of a screen displayed in the information processing of FIG. 7.

The process in FIG. 7 is triggered when a user logs in to the skin condition estimation service provided by the server 30 using user identification information.

As shown in FIG. 7, the client apparatus 10 executes estimation request (S1110).

Specifically, the processor 12 displays screen P1110 (FIG. 8) on the display.

The screen P1110 includes an operation object B1110 and a field object F1110.

The operation object B1110 is an object that receives a user instruction for finalizing an input to the field object F1110.

The field object F1110 is an object that accepts input of mind-body data.

When the user inputs mind-body data into the field object F1110 and operates the operation object B1110, the processor 12 transmits estimation request data to the server 30.

The estimation request data includes, for example, the following information:
Mind-body data entered in the field object F1110.

After step S1110, the server 30 executes estimating skin condition (S1130).

Specifically, the processor 32 provides the mind-body data X included in the estimation request data to the skin model M, and estimates the skin condition Y corresponding to the mind-body data X.

More specifically, the processor 32 estimates the skin condition Y1 of the skin index “skin moisture” by providing the mind-body data X included in the estimation request data to the skin model M1.

The processor 32 estimates the skin condition Y2 of the skin index “barrier strength” by providing the mind-body data X included in the estimation request data to the skin model M2.

The processor 32 estimates the skin condition Y3 of the skin index “skin texture” by providing the mind-body data X included in the estimation request data to the skin model M3.

The processor 32 estimates the skin condition Y4 of the skin index “skin firmness” by providing the mind-body data X included in the estimation request data to the skin model M4.

The processor 32 estimates the skin condition Y5 of the skin index “skin wrinkles” by providing the mind-body data X included in the estimation request data to the skin model M5.

The processor 32 estimates the skin condition Y6 of the skin index “skin melanin” by providing the mind-body data X included in the estimation request data to the skin model M6.

The processor 32 estimates the skin condition Y7 of the skin index “skin hemoglobin” by providing the mind-body data X included in the estimation request data to the skin model M7.

The processor 32 estimates the skin condition Y8 of the skin index “skin brightness” by providing the mind-body data X included in the estimation request data to the skin model M8.

The processor 32 estimates the skin condition Y9 of the skin index “skin vividness” by providing the mind-body data X included in the estimation request data to the skin model M9.

The processor 32 estimates the skin condition Y10 of the skin index “skin redness” by providing the mind-body data X included in the estimation request data to the skin model M10.

The processor 32 estimates the skin condition Y11 of the skin index “yellowing of the skin” by providing the mind-body data X included in the estimation request data to the skin model M11.

The processor 32 estimates the skin condition Y12 of the skin index “skin spots” by providing the mind-body data X included in the estimation request data to the skin model M12.

After step S1130, the server 30 executes generating advice (S1131).

Specifically, the memory 31 stores an advice model.

The advice model describes the correlation between the skin condition and the advice.

The processor 32 inputs the estimation result of the skin condition acquired in step S1130 into the advice model, thereby generating advice according to the estimation result of the skin condition.

After step S1131, the server 30 executes updating database (S1132).

Specifically, the processor 32 adds a new record to the inferred log database (FIG. 5) associated with the subject identification information used to log in.

The following information is stored in each field of the new record:
“estimation log ID” field: New estimation log identification information;
“estimation date and time” field: information on the date and time of execution of step S1130;
“estimation result” field: Estimation result information acquired in step S1130; and
“advice” field: Advice information acquired in step S1131.

After step S1132, the server 30 executes estimation response (S1133).

Specifically, the processor 32 transmits the estimation response data to the client apparatus 10.

The estimation response data includes, for example, the following information:
Estimation result of the skin condition acquired in step S1130; and
Advice acquired in step S1131.

After step S1133, the client apparatus 10 executes outputting estimation result (S1111).

Specifically, the processor 12 displays screen P1111 (FIG. 8) on the display.

Screen P1111 includes display objects A1111a to A1111b.

The display object A 1111a displays the estimation result of the skin condition included in the estimation response data.

The display object A 1111b displays advice included in the estimation response data.

### (6) Summary of the Present Embodiment

According to the present embodiment, by providing a subject's mind-body data to a model describing the correlation between the mind-body data and the skin condition, the skin condition corresponding to the mind-body data of the subject is estimated.

This allows for a more appropriate estimation of the skin condition and the factors that define the skin condition.

According to the present embodiment, the influence degree between the mind-body data and the skin index may be defined for each combination of the mind-body data and the skin index.

This allows for a more appropriate estimation of the skin condition and the factors that define the skin condition.

According to the present embodiment, advice may be output according to the estimated skin condition.

This makes it possible to provide the subject with information that is useful for improving their skin condition.

### (7) Modification

A modification of the present embodiment will now be described.

### (7-1) First Modification

The first modification will be described.

The first modification is an example that uses a skin model in which the influence of human attributes is reduced.

For example, it can be said that the barrier function of men's cheek skin is weaker than that of women, it contains more oil than women, and it has less elasticity than women.

This may be due to the difference in the amount of male and female hormones secreted.

For example, as we age, the skin can become wrinkled, less elastic, and less hydrated.

This is because aged cells that form inside the skin as we age reduce the function of surrounding cells, causing aging changes in the skin.

In this way, there is a correlation between a person's attributes and their skin condition.

If a person's attributes are taken into excessive consideration, the estimation result of the skin condition will be excessively influenced by the attributes in cases where the skin condition is actually determined by factors other than the attributes.

Therefore, corrections taking into account the attributes of the subjects may be necessary.

### (7-1-1) Overview of First Modification

The overview of the first modification will be described.

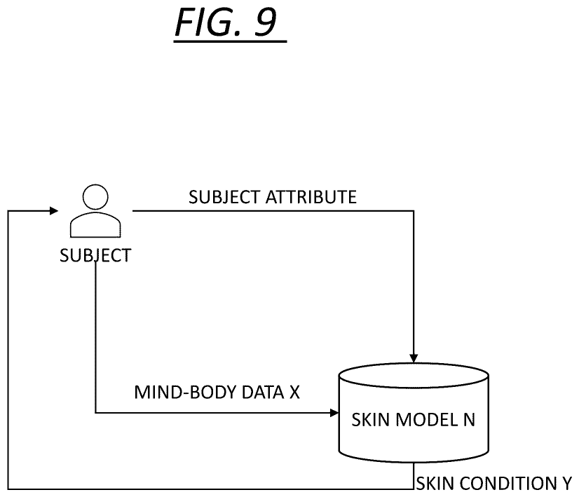

FIG. 9 is a diagram illustrating an overview of the first modification.

As shown in FIG. 9, in the skin model N of the first modification, the influence of the skin model M of the present embodiment is adjusted taking into account human attributes.

When the subject's mind-body data X (including the subject's attributes) is provided to a skin model N, a skin condition Y corresponding to the mind-body data X is estimated so that the influence of the subject's attributes is not excessively expressed (for example, at least one of the influences of deterioration due to aging and the influences of deterioration due to gender is excluded).

The estimated skin condition Y is presented to the subject.

### (7-1-2) Information Processing of First Modification

The information processing of the first modification will be described.

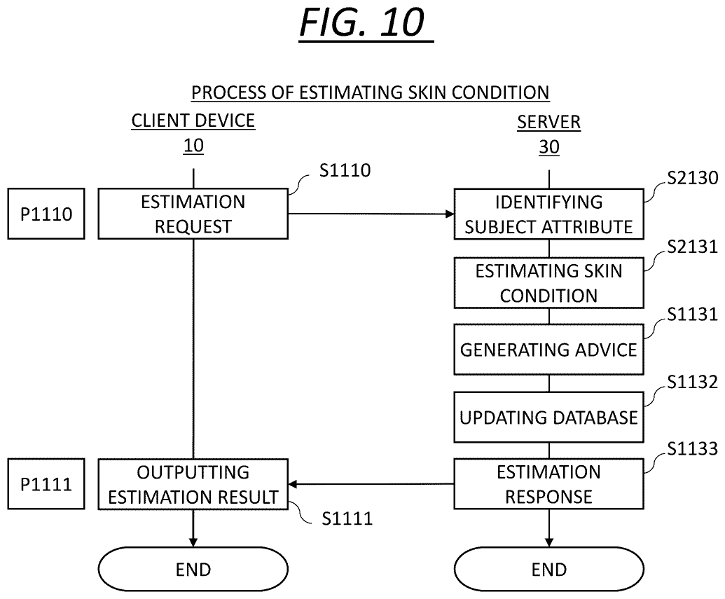

FIG. 10 is a sequence diagram of information processing according to the first modification.

The trigger for the process in FIG. 10 is the same as that in FIG. 7.

As shown in FIG. 10, the client apparatus 10 executes the estimation request (S1110) in the same manner as in FIG. 7.

After step S1110, the server 30 executes identifying subject attribute (S2130).

In a first example of step S2130, the target attribute is age.

Specifically, the processor 32 references the subject database (FIG. 4) to identify the birthday information associated with the subject identification information.

The processor 32 calculates the age of the subject at the time step S2130 is executed based on the birthday information and the date and time when step S2130 is executed.

In a second example of step S2130, the subject attribute is gender.

Specifically, the processor 32 refers to the subject database (FIG. 4) to identify the gender information associated with the subject identification information.

After step S2130, the server 30 executes estimating skin condition (S2131).

Specifically, the processor 32 provides the mind-body data X included in the estimation request data and the subject attributes (at least one of age and gender) acquired in step S2130 to a skin model N, and estimates a skin condition Y according to the combination of the mind-body data X and the subject attributes.

After step S2131, the server 30 executes generating advice (S1131), updating database (S1132), and estimation response (S1133), in the same manner as in FIG. 7.

After step S1133, the client apparatus 10 executes outputting estimation result (S1111) in the same manner as in FIG. 7.

### (7-1-3) Summary of First Modification

According to the first modification, the skin condition can be estimated so that the influence of subject attributes (for example,, at least one of age and gender) is not excessively expressed.

This makes it possible to present an estimation result of the skin condition according to the subject's age.

As a result, the skin condition and the factors that define the skin condition can be more appropriately estimated.

### (7-2) Second Modification

The second modification will now be described.

The second modification is an example in which a skin model in which the influence of the subject's body composition is reduced is used.

For example, obesity is known to increase water loss, slow collagen production, increase sebum secretion, and restrict lymphatic flow.

In this way, there is a correlation between body composition and skin condition.

Therefore, it is necessary to take into account the influence of body composition such as body size, fat mass, and BMI, or the like.

### (7-2-1) Overview of Second Modification

The overview of the second modification will now be described.

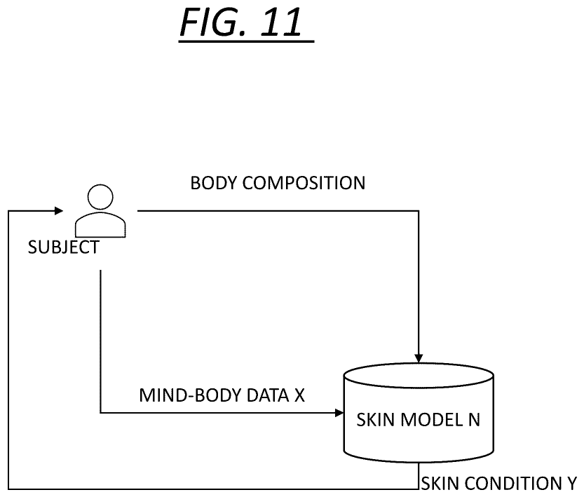

FIG. 11 is a diagram illustrating an overview of the second modification.

As shown in FIG. 11, in the skin model N of the second modification, the influence of the skin model M of the present embodiment is adjusted in consideration of the body composition.

When the subject's mind-body data X (including the subject's body composition) is provided to the skin model N, a skin condition Y corresponding to the mind-body data X is estimated so that the influence of the subject's body composition is not excessively expressed (for example,, the influence of deterioration due to obesity is excluded).

The body composition may be, for example, at least one of the following:
Muscle mass per unit weight;
Body fat mass;
Muscle mass;
BMI;
Body fat mass for each part (for example, arm, leg, and core);
Muscle mass for each part (for example, arm and leg);
Moisture level for each part (for example, arm, leg, and core);
Skeletal muscle mass;
Basal metabolic rate; and
Fat-free mass.

When the subject's mind-body data X (including the subject's body composition) is provided to a skin model N, a skin condition Y corresponding to the mind-body data X is estimated.

The estimated skin condition Y is presented to the subject.

### (7-2-2) Information Processing of Second Modification

The information processing of the second modification will be described.

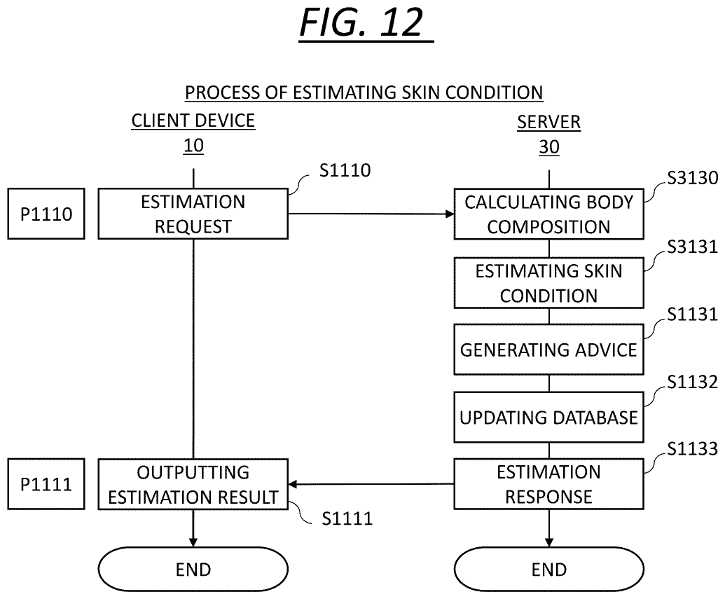

FIG. 12 is a sequence diagram of information processing according to the second modification.

The trigger for the process in FIG. 12 is the same as that in FIG. 7.

As shown in FIG. 12, the client apparatus 10 executes estimation request (S1110) in the same manner as in FIG. 7.

After step S1110, the server 30 executes calculating body composition (S3130).

Specifically, the processor 32 calculates the body composition based on the mind-body data included in the estimation request data.

After step S3130, the server 30 executes estimating skin condition (S3131).

Specifically, the processor 32 provides the mind-body data X included in the estimation request data to a skin model N, and estimates a skin condition Y corresponding to the mind-body data X.

After step S3131, the server 30 generating advice (S1131), updating database (S1132), and estimation response (S1133), in the same manner as in FIG. 7.

After step S1133, the client apparatus 10 executes outputting estimation result (S1111) in the same manner as in FIG. 7.

### (7-2-3) Summary of Second Modification

According to the second modification, the skin condition can be estimated without excessively being influenced by the subject's body composition.

This allows the skin condition and the factors that define the skin condition to be more appropriate.

### (7-3) Third Modification

The third modification will now be described.

The third modification is an example in which two types of skin models are used depending on the invasiveness of the mind-body data.

### (7-3-1) Overview of Third Modification

The overview of the third modification will now be described.

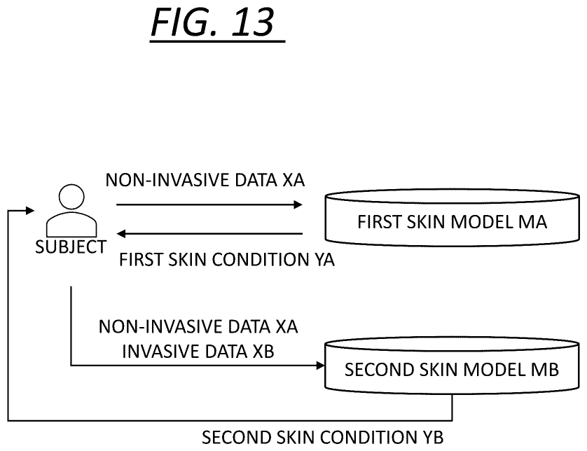

FIG. 13 is a diagram illustrating an overview of the third modification.

As shown in FIG. 13, the skin model of the third modification example includes the first skin model MA and the second skin model MB.

The first skin model MA describes the correlation between the non-invasive data XA that can be acquired by a non-invasive test and the first skin condition YA.

The second skin model MB describes the correlation between the invasive data XB and non-invasive data XA that can be acquired by an invasive test and the second skin condition YB.

In other words, the second skin model MB is the same as to the skin model M of the present embodiment.

When the subject's mind-body data X is provided to the first skin model MA, a first skin condition YA corresponding to the non-invasive data XA of the subject's mind-body data X is estimated.

### The Estimated First Skin Condition Ya Is Presented to the Subject.

When the subject's mind-body data X is provided to the second skin model MB, a second skin condition YB corresponding to the mind-body data X (that is, the non-invasive data XA and the invasive data XB) is estimated.

The estimated second skin condition YB is presented to the subject.

The number of parameters given to the second skin model MB for estimating the second skin condition YB is greater than the number of parameters given to the first skin model MA for estimating the first skin condition YA.

Therefore, the estimation accuracy of the second skin model MB is higher than the estimation accuracy of the first skin model MA.

### (7-3-2) Skin Model of Third Modification

The skin model of the third modification will be described.

### (7-3-2-1) Configuration of First Skin Model of Third Modification

The configuration of the first skin model of the third modification will be described.

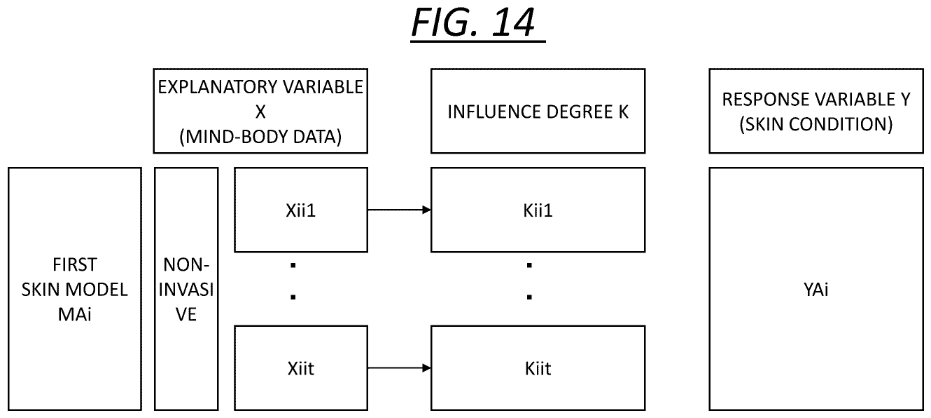

FIG. 14 is a schematic diagram showing a configuration of the first skin model of the third modification.

As shown in FIG. 14, an explanatory variable Xiit, an influence degree Kiit, and a response variable YAi are defined for the first skin model MAi.

The argument t (t is a natural number) is an identifier of mind-body data (hereinafter referred to as “non-invasive data”) collected by a non-invasive test.

The explanatory variable Xiit is a variable related to the non-invasive data.

The influence degree Kiit indicates the influence degree of the non-invasive data on the skin condition.

The objective variable YAi is the skin condition.

### (7-3-2-2) Configuration of Second Skin Model of Third Modification

The configuration of the second skin model in the third modification will be described.

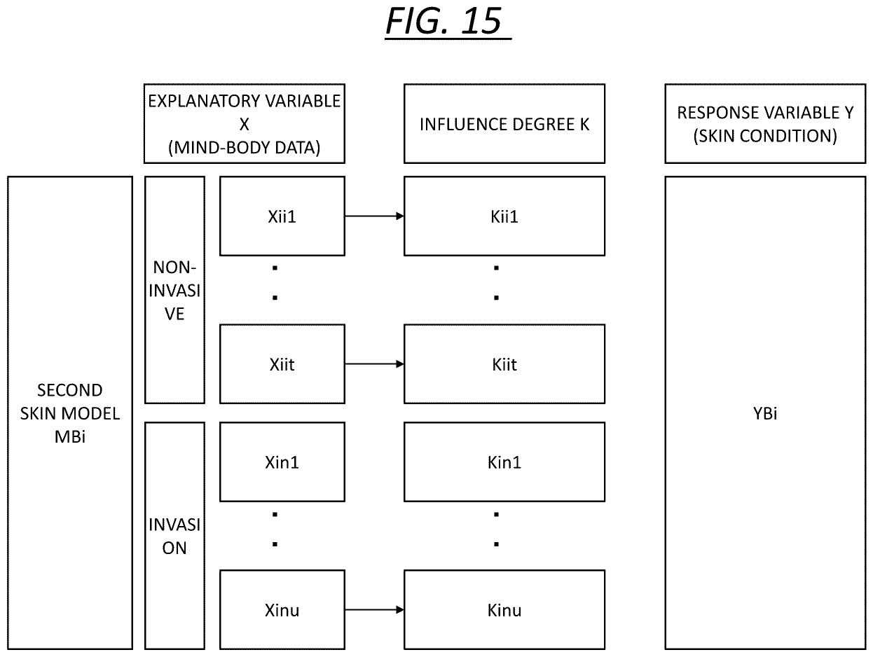

FIG. 15 is a schematic diagram showing the configuration of the second skin model of the third modification.

As shown in FIG. 15, the second skin model MBi is defined with an explanatory variable Xiit, an influence Kiit, a response variable YBi, an explanatory variable Xinu (u is a natural number), an influence Kiiu, and a response variable YBi.

As shown in FIG. 15, an explanatory variable Xinu, an influence degree Kinu, and a response variable YBi are defined for the second skin model MBi.

The argument u (u is a natural number) is an identifier of mind-body data (hereinafter referred to as “invasive data”) collected by an invasive examination (hereinafter referred to as “invasive examination”).

The explanatory variable Xinu is a variable related to the invasive data.

The influence degree Kinu indicates the influence degree that the invasive data has on the skin condition.

The objective variable YBi is the skin condition.

### (7-3-3) Information Processing of Third Modification

The information processing of the third modification will be described.

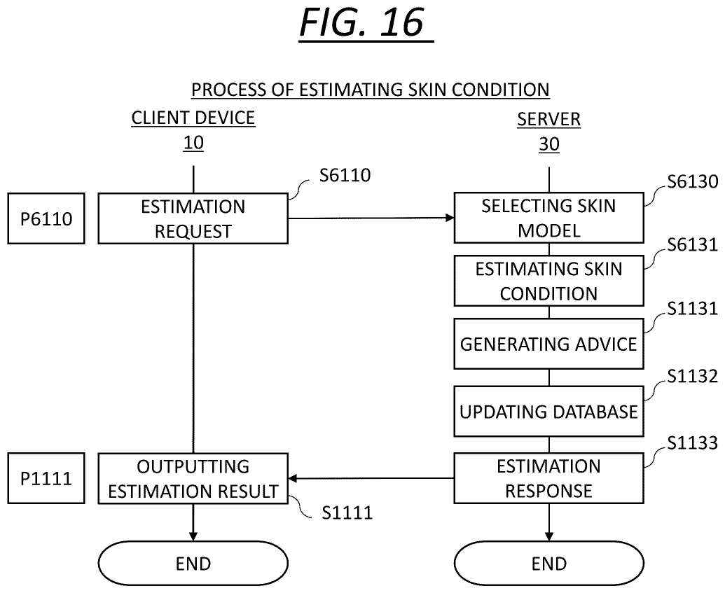

FIG. 16 is a sequence diagram of information processing according to the third modification.

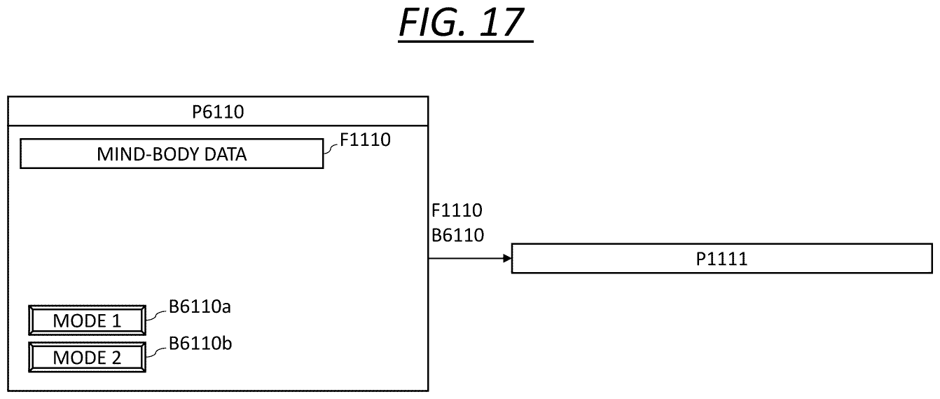

FIG. 17 is a diagram showing an example of a screen displayed in the information processing of FIG. 16.

The trigger for the process in FIG. 16 is the same as that in FIG. 7.

As shown in FIG. 16, the client apparatus 10 executes estimation request (S6110).

Specifically, the processor 12 displays screen P6110 (FIG. 17) on the display.

The screen P6110 includes operation objects B6110a to B6110b and a field object F1110.

The operation object B6110a is an object that confirms an input to the field object F1110 and receives a user instruction for requesting estimation in the first estimation mode.

The first estimation mode is a mode in which estimation is performed using the first skin model.

The operation object B6110a is assigned mode identification information “MODE1” that identifies the first mode.

The operation object B6110b is an object that confirms an input to the field object F1110 and receives a user instruction for requesting estimation in the second estimation mode.

The second estimation mode is a mode in which estimation is performed using the second skin model.

The operation object B6110b is assigned mode identification information “MODE2” that identifies the second mode.

The field object F1110 is the same as that in FIG. 8.

When the user inputs mind-body data into the field object F1110 and operates the operation object B6110a or B6110b, the processor 12 transmits estimation request data to the server 30.

The estimation request data includes, for example, the following information:
Mind-body data entered in the field object F1110; and
Mode identification information “MODE1” or “MODE2” corresponding to the operated operation object B6110a or B6110b.

After step S6110, the server 30 executes selecting skin model (S6130).

Specifically, the memory 31 stores the first skin model MAi and the second skin model MBi.

The first skin model MAi corresponds to the mode identifying information “MODE1”.

The second skin model MBi corresponds to the mode identifying information “MODE2”.

The processor 32 selects a skin model (the first skin model MAi or the second skin model MBi) corresponding to the mode identification information included in the estimation request data.

After step S6130, the server 30 executes estimating skin condition (S6131).

Specifically, when the first skin model MAi is selected in step S6130, the processor 32 provides the mind-body data X (hereinafter referred to as “non-invasive data”) that is assigned the label “non-invasive” from the mind-body data X included in the estimation request data to the first skin model MAi selected in step S6130, thereby estimating the first skin condition YAi corresponding to the non-invasive data.

When the second skin model MBi is selected in step S6130, the processor 32 provides the mind-body data X which have non-invasive data that is assigned the label “non-invasive” and invasive data that is assigned the label “invasive data” to the first skin model MAi selected in step S6130, thereby estimating the second skin condition YBi corresponding to the mind-body data X.

After step S6131, the server 30 executes generating advice (S1131), updating database (S1132), and estimation response (S1133), in the same manner as in FIG. 7.

After step S1133, the client apparatus 10 executes outputting estimation result (S1111) in the same manner as in FIG. 7.

### (7-3-4) Summary of Third Modification

According to the third modification, the determination using the first skin model MAi and the determination using the second skin model MBi are switched in response to an instruction from the subject.

The first skin model MAi is configured to estimate the skin condition using non-invasive data of the subject, and therefore imposes less of a burden on the subject for estimating the skin condition than the second skin model MBi.

The second skin model MBi is configured to estimate the skin condition using non-invasive data and invasive data of the subject, and therefore has higher estimation accuracy than the first skin model MAi.

This makes it possible to provide skin condition estimation results using an appropriate skin model tailored to the needs of the subject.

### (7-4) Fourth Modification

### The Fourth Modification Will Now Be Described.

The fourth modification is an example in which a skin model in which the influence of the subject's habits is reduced is used.

For example, people who are more active in the morning or evening, differ in the peak rise in body temperature and the secretion of cortisol and melatonin in the endocrine system.

Furthermore, those people also differ in the circadian rhythm of blood factors. For example, exposure to ultraviolet rays causes collagen decomposition and melanin secretion, resulting in changes in skin elasticity and color.

In this way, there is a correlation between a person's habits and their skin condition.

If a person's habits are taken into excessive consideration, the estimation results of the skin condition will be overly influenced by habits in cases where the skin condition is actually determined by factors other than habits.

### (7-4-1) Overview of Fourth Modification

The overview of the fourth modification will now be described.

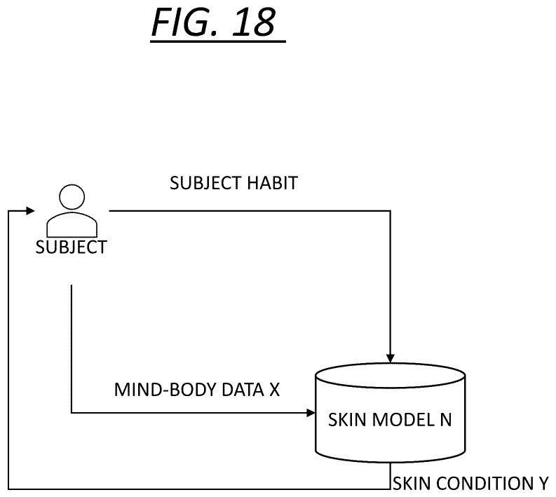

FIG. 18 is a diagram illustrating an overview of the fourth modification.

As shown in FIG. 18, in the skin model N of the fourth modification, the influence defree of the skin model M of the present embodiment is adjusted taking into account human habits.

When the subject's mind-body data X and the subject's habits are provided to a skin model N, a skin condition Y corresponding to the mind-body data X is estimated so that the influence degree of the subject's habits is not excessively expressed.

The estimated skin condition Y is presented to the subject.

For example, the subject's habits include at least one of the following:
Sleeping habits (for example, morning person or night person); and
Lifestyle habits (for example, tendency for ultra violet exposure).

### (7-4-2) Information Processing of Fourth Modification

The information processing of the fourth modification will be described.

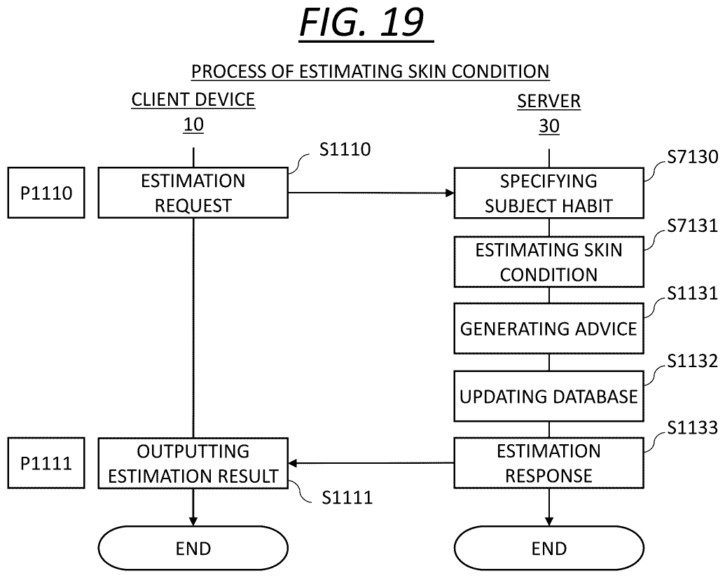

FIG. 19 is a sequence diagram of information processing according to the fourth modification.

The trigger for the process in FIG. 19 is the same as that in FIG. 7.

As shown in FIG. 19, the client apparatus 10 executes the estimation request (S1110) in the same manner as in FIG. 7.

After step S1110, the server 30 executes specifying subject habit (S7130).

Specifically, the memory 31 stores subject habit information (not shown) in association with subject identification information.

The subject habit information is information on the subject's habits.

The subject habit information is acquired, for example, by at least one of the following methods:
Questionnaire for the target audience;
Wearable device worn by the subject; and
Mobile phone (for example, smart phone) which the subject brings.

The processor 32 determines subject habit information associated with the subject identification information.

The processor 32 identifies the subject's habits at the time of execution of step S7130 based on the subject's habit information and the execution date and time of step S2130.

After step S7130, the server 30 executes estimating skin condition (S7131).

Specifically, the processor 32 provides the mind-body data X included in the estimation request data and the subject habit information acquired in step S7130 to a skin model N, thereby estimating a skin condition Y according to the combination of the mind-body data X and the subject habit information.

After step S7131, the server 30 executes generating advice (S1131), updating database (S1132), and estimation response (S1133), in the same manner as in FIG. 7.

After step S1133, the client apparatus 10 executes outputting estimation result (S1111) in the same manner as in FIG. 7.

### (7-4-3) Overview of Fourth Modification

According to the fourth modification, the skin condition can be estimated without being excessively influenced by the subject's habits.

This allows the skin condition and the factors that define the skin condition to be more appropriately estimated.

### (7-5) Fifth Modification

### The Fifth Modification Will Now Be Described.

The fifth modification is an example in which a skin model in which the influence of the estimation time is reduced is used.

For example, the skin is greatly affected by ultraviolet rays, humidity, and temperature, and the moisture content, skin color, and sebum secretion amount change depending on the season.

In addition, since the body's metabolism and vitamin production are also affected by temperature and ultraviolet rays, the condition of the skin changes with the seasons.

As such, there is a correlation between time and skin condition.

If the time period is taken into excessive consideration, the estimation result of the skin condition will be excessively influenced by the time period in cases where the skin condition is actually determined by factors other than the time period.

### (7-5-1) Overview of Fifth Modification

The overview of the fifth modification will now be described.

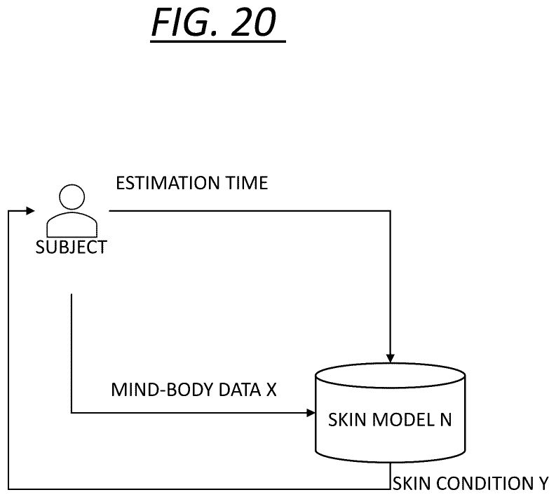

FIG. 20 is a diagram illustrating an overview of the fifth modification.

As shown in FIG. 20, in the skin model N of the fifth modification, the influence of the skin model M of the present embodiment is adjusted taking into account the estimation time.

The estimation time is a time period that is specified from the date on which the skin condition estimation is performed.

When the subject's mind-body data X is provided to a skin model N, a skin condition Y corresponding to the mind-body data X is estimated so that the influence of the estimation time is not excessively expressed.

The estimated skin condition Y is presented to the subject.

For example, the estimation time includes at least one of the following:
Season estimated from the date of skin condition estimation (for example, spring, summer, autumn, winter); and
The date, month, or day on which the skin condition estimation is to be executed.

### (7-5-2) Information Processing of Fifth Modification

The information processing of the fifth modification will be described.

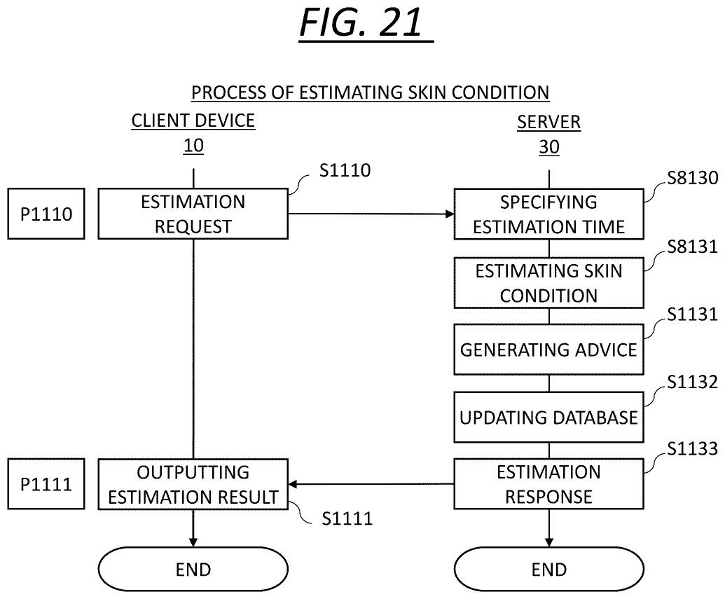

FIG. 21 is a sequence diagram of information processing according to the fifth modification.

The trigger for the process in FIG. 21 is the same as that in FIG. 7.

As shown in FIG. 21, the client apparatus 10 executes the estimation request (S1110) in the same manner as in FIG. 7.

After step S1110, the server 30 executes the specifying estimation time (S8130).

Specifically, the memory 31 stores rules for specifying the estimation time (hereinafter, referred to as “estimation time specification rule”).

The estimation time specification rule describes the correspondence between the execution date and time of step S8130 and the estimation time.

The processor 32 specifies the estimation time corresponding to the execution date and time of step S8130 by providing the execution date and time of step S8130 (that is, the timing at which the skin condition is estimated) to the estimation time specification rule.

After step S8130, the server 30 executes estimating skin condition (S8131).

Specifically, the processor 32 provides the mind-body data X included in the estimation request data and the estimation time acquired in step S8130 to a skin model N, and estimates a skin condition Y corresponding to the combination of the mind-body data X and the estimation time.

After step S8131, the server 30 executes generating advice (S1131), updating database (S1132), and estimation response (S1133), in the same manner as in FIG. 7.

After step S1133, the client apparatus 10 executes outputting estimation result (S1111) in the same manner as in FIG. 7.

### (7-5-3) Summary of Fifth Modification

According to the fifth modification, the skin condition can be estimated without being excessively affected by the estimation time.

This allows the skin condition and the factors that define the skin condition to be more appropriately estimated.

### (8) Other Modifications

Other modifications will now be described.

The memory 11 may be connected to the client apparatus 10 via a network NW.

The memory 31 may be connected to the server 30 via a network NW.

Each step of the above information processing can be executed by either the client apparatus 10 or the server 30.

For example, if the client apparatus 10 is capable of executing all the steps of the above-mentioned information processing, the client apparatus 10 functions as an information processing apparatus that operates standalone without transmitting requests to the server 30.

In the present embodiment, an example of displaying a screen on a display has been given as an example of output, but the scope of the present embodiment is not limited to this.

This embodiment is also applicable to the following examples:
Sound output of the estimation result;
Print output of the estimation result; and
Light output of the estimation result.

Although the present embodiments of the present invention are described in detail above, the scope of the present invention is not limited to the above embodiments. Further, various modifications and changes can be made to the above embodiments without departing from the spirit of the present invention.

In addition, the above embodiments and variations can be combined.

### REFERENCE SIGNS LIST

1: Information processing system
10: Client apparatus
11: Memory
12: Processor
13: Input and output interface
14: Communication interface
30: Server
31: Memory
32: Processor
33: Input and output interface
34: Communication interface

1. A method for estimating a skin condition, the method comprising the steps of:
acquiring mind-body data relating to the body and/or mind of a subject of skin condition estimation; and
estimating the subject's skin condition using a skin model that describes a correlation between the mind-body data and the skin condition, the mind-body data including at least one of liver function related variables, muscle-bone related variables, heart rate and blood pressure related variables, blood oxidation related variables, blood count related variables, lipid metabolism related variables, autonomic function related variables, personal attribute related variables, renal function related variables, body composition related variables, glucose tolerance related variables, electrolyte related variables, cognitive function related variables, immunometabolism related variables, and depression-fatigue-drowsiness related variables.

2. The method according to claim 1, wherein the liver function related variables include at least one of ALT (GPT) in blood, AST (GOT) in blood, ALP in blood, γ-GTP in blood, indirect bilirubin in blood, total bilirubin in blood, and direct bilirubin in blood.

3. The method according to claim 1, wherein the muscle-bone related variables include at least one of grip strength and bone density.

4. The method according to claim 1, the heart rate and blood pressure related variable includes at least one of diastolic blood pressure, systolic blood pressure, and heart rate.

5. The method according to claim 1, the blood oxidation related variables include at least one of biological antioxidant potential in blood, reactive oxygen metabolites in blood, and oxidation stress Index.

6. The method according to claim 1, wherein blood count related variables include at least one of hematocrit in blood, hemoglobin in blood, platelets in blood, red blood cell in blood, white blood cell in blood, mean corpuscular hemoglobin concentration in blood, mean corpuscular hemoglobin in blood, and mean corpuscular volume in blood.

7. The method according to claim 1, wherein the lipid metabolism related variables include at least one of HDL cholesterol in blood, LDL cholesterol in blood, total cholesterol in blood, a ratio of LDL cholesterol in blood to HDL cholesterol in blood, and neutral fat in blood.

8. The method according to claim 1, wherein the autonomic function related variables include at least one of autonomic indices HF, autonomic indices LF, and a ratio of the autonomic indices HF to the autonomic indices LF.

9. The method according to claim 1, wherein the personal attribute related variables include at least one of exercise habits, sleep time value, duration of fatigue, average working time, medical history, smoking history, regular medication, and marital history.

10. The method according to claim 1, wherein the renal function related variables include at least one of estimated glomerular filtration rate and urea nitrogen in blood.

11. The method according to claim 1, wherein the body composition related variables include at least one of BMI (Body Mass Index), muscle mass, height, body fat percentage, skeletal muscle mass, and body weight.

12. The method according to claim 1, wherein the glucose tolerance related variables include at least one of hemoglobin A1c (HbA1c) in blood and glucose in blood.

13. The method according to claim 1, wherein the electrolyte related variables include at least one of kalium in blood, chloride in blood, a ratio of natrium in blood to kalium in blood, and natrium in blood.

14. The method according to claim 1, wherein the cognitive function related variables include at least one of a reaction time of cognitive tasks, a correct answer rate of cognitive tasks, an incorrect answer rate of cognitive tasks, and a number of completed cognitive tasks.

15. The method according to claim 1, wherein the immunometabolism related variables include at least one of high-sensitivity CRP in blood, amylase in blood, albumin in blood, creatine kinase in blood, cortisol in blood, total protein in blood, lactate dehydrogenase in blood, IgE in blood, IgG in blood, and uric acid in blood.

16. The method according to claim 1, wherein the depression-fatigue-sleepiness related variables include at least one of fatigue assessment scale, depression-anxiety assessment scale, motivation assessment scale, drowsiness assessment scale, and life performance status assessment scale.

17. The method according to claim 1, wherein the step of estimating the skin condition estimates skin moisture with reference to a combination of the body composition related variable, the blood oxidation related variable, the blood count related variable, the depression-fatigue-drowsiness related variable, the electrolyte related variable, the glucose tolerance related variable, and the immunometabolism related variable.

18. The method according to claim 1, wherein the step of estimating the skin condition estimates a barrier strength of the skin with reference to a combination of the electrolyte related variable, the liver function related variable, the glucose tolerance related variable, the blood oxidation related variable, the blood count related variable, the glucose tolerance related variable, and the depression-fatigue-sleepiness related variable.

19. The method according to claim 1, wherein the step of estimating the skin condition estimates a skin texture with reference to a combination of the immunometabolism related variable, the electrolyte related variable, the autonomic function related variable, and the liver function related variable.

20. The method according to claim 1, wherein the step of estimating the skin condition estimates skin firmness with reference to a combination of the blood oxidation related variable, the liver function related variable, the immunometabolism related variable, the blood oxidation related variable, the body composition related variable, the glucose tolerance related variable, the blood count related variable, the muscle-bone related variable, and the autonomic function related variable.

21. The method according to claim 1, wherein the step of estimating the skin condition estimates skin melanin with reference to a combination of the autonomic function related variable, the depression-fatigue-drowsiness related variable, the immunometabolism related variable, the glucose tolerance related variable, the liver function related variable, the blood count related variable, and the blood oxidation related variable.

22. The method according to claim 1, wherein the step of estimating the skin condition estimates skin brightness with reference to a combination of the body composition related variables, the blood count related variables, the renal function related variables, the immunometabolism related variables, the heart rate and blood pressure related variables, the lipid metabolism related variables, the electrolyte related variables, and the depression-fatigue-drowsiness related variables;

23. The method according to claim 1, wherein the step of estimating the skin condition estimates redness of the skin with reference to a combination of the body composition related variable, the depression-fatigue-drowsiness related variable, the blood count related variable, the liver function related variable, the blood count related variable, the blood oxidation related variable, and the immunometabolism related variable.

24. (canceled)

25. The method according to claim 1, wherein the step of estimating estimates the skin condition by providing mind-body data including the attributes of the subject to a skin model in which an influence degree of at least one of a person's attributes, a person's body composition, a person's habits, and a time is reduced.

26. The method according to claim 25, wherein the attribute of the subject is age or gender.

27. (canceled)

28. The method according to claim 1, wherein the skin model includes a first skin model generated using only non-invasive data measured by a non-invasive test that does not involve invasiveness, and a second skin model generated using invasive data measured by an invasive test that involves invasiveness and the non-invasive data.

29. (canceled)

30. (canceled)

31. (canceled)
